(19) United States
(12) Patent Application Publication
(10) Pub. No.: US 2021/0220437 A1
(43) Pub. Date: Jul. 22, 2021

(54) Compositions and Methods Relating to p62 for the Treatment and Prophylaxis of Age-Related Macular Degeneration

(71) Applicant: CureLab Oncology, Inc., DEDHAM, MA (US)

(72) Inventors: Alexander Shneider, DEDHAM, MA (US); Michael Sherman, NEWTON, MA (US); Vladimir Gabai, BRIGHTON, MA (US); Nataliya Kolosova, Novosibirsk (RU); Oyuna Kozhevnikova, Novosibirsk (RU)

(21) Appl. No.: 17/265,516

(22) PCT Filed: Oct. 2, 2019
(86) PCT No.: PCT/US2019/054370

§ 371 Date: Feb. 2, 2021

Related U.S. Application Data
(60) Provisional application No. 62/713,544, filed on Aug. 2, 2018.

(51) Int. Cl. A61K 38/17; A61K 48/00; A61P 27/02

(52) U.S. Cl. CPC A61K 38/1709; A61P 27/02; A61K 48/0066

(57) ABSTRACT

Novel p62/SQSTM1 compositions for the prophylaxis and treatment of agerelated macular degeneration. Modified p62 compositions and methods to increase activity of p62 for such prophylaxis and treatment.

### RELATED APPLICATIONS

This application claims the benefit under 35 USC 119(e) of U.S. Provisional Applications No. 62/713,544, filed on Aug. 2, 2018, which is incorporated herein by reference in its entirety.

### FIELD OF THE INVENTION

The invention relates generally to prevention and treatment of age-related degenerative diseases. More specifically, the invention relates to prevention, treatment, and prophylaxis of age-related macular degeneration using p62-based compositions and medications.

### BACKGROUND

Age-related macular degeneration (AMO) is the most common cause of irreversible vision loss in industrialized countries. Prevalence of AMO is increasing dramatically as the proportion of the elderly in the population continues to rise. By clinical signs, there are two forms of AMO: dry and wet AMO forms, also known as geographic atrophy and exudative AMO, respectively. There are effective treatments of vascular complications of neovascular AMO by anti-VEGF therapeutics. However, neither there is a treatment of the dry form of AMO (˜90% of all cases) nor preventive strategies against progression to the non-exudative form of AMO. Therefore, the development of effective therapeutic and prophylactic modalities against AMO is an urgent task. AMO is a multifactorial disease involving a complex interplay of genetic, environmental, metabolic, and functional factors.

The adapter protein p62/SQSTM1 interacts with many signaling factors, and regulates major cellular functions including apoptosis autophagy, and apoptosis. FIG. 1 shows the nucleic acid sequence of the cDNA and FIG. 2 the amino acid sequence of the encoded p62 protein used in various studies. In the retinal pigment epithelium (RPE), p62 promotes autophagy and simultaneously enhances a Nrf2-mediated antioxidant response to protect against acute oxidative stress. Recently, a DNA plasmid encoding p62-SQSTM1 (p62DNA) demonstrated strong anti-osteoporotic activity and alleviated diet-induced obesity and metabolic dysfunctions in animal models

### SUMMARY

Disclosed herein is an agent to treat, alleviate, ameliorate, relieve, delay onset of, inhibit progression of, reduce severity of, and/or reduce incidence of one or more symptoms of age-related macular degeneration by administering to the subject an agent having: at least 30 consecutive amino acids of a p62/SQSTM1 polypeptide; a p62/SQSTM1 encoding nucleic acid, wherein said p62/SQSTM1 encoding nucleic acid encodes at least 30 consecutive amino acids of a p62/SQSTM1 polypeptide; a p62/SQSTM1 polypeptide at least 90% identical to SEQ ID NO. 2; a p62/SQSTM1 polypeptide with at least one or more domain deletions; a p62/SQSTM1 nucleic acid that encodes a polypeptide at least 90% identical to SEQ ID NO. 2 or a p62/SQSTM 1 nucleic acid that encodes a polypeptide with at least one or more domain deletions.

In some embodiments, the said agent is for administration with an agent used to prevent or treat age-related macular degeneration.

In some embodiments, the use of the above agent also in preventing, reversing, reducing or modulating intraocular vascularization, oxidative stress, and autophagy.

In some embodiments, the said agent is administered via a carrier such as microorganism, virus, nanoparticle, polymer, liposome, protein, and via intraocular, intramuscular, subcutaneous, per oral, per rectum, intranasal, intradermal routs of administering.

In some embodiments, the use of said agent for patients which are selected based on signs of age-related macular degeneration.

Also disclosed is a method of preventing, treating, alleviating, ameliorating, relieving, delaying onset of, inhibiting progression of, reducing severity of, or reducing incidence of one or more symptoms of age-related macular degeneration in a subject comprising administering to the subject an agent comprising: at least 30 consecutive amino acids of a p62/SQSTM1 polypeptide or a variant thereof; a p62/SQSTM1 encoding nucleic acid, wherein said p62/SQSTM1 encoding nucleic acid encodes at least 30 consecutive amino acids of a p62/SQSTM1 polypeptide or a variant thereof; a p62/SQSTM1 polypeptide at least 90% identical to SEQ ID NO. 2; a p62/SQSTM1 polypeptide with at least one domain deletion; a p62/SQSTM1 nucleic acid encoding a polypeptide at least 90% identical to SEQ ID NO. 2; or a p62/SQSTM1 nucleic acid encoding a polypeptide with at least one domain deletion.

In some embodiments, the administering the agent to the subject is performed in combination with a second agent preventing or treating age-related macular degeneration.

Also disclosed is a method of preventing, reversing, reducing, or modulating intraocular vascularization, oxidative stress, autophagy, or inflammation in a subject comprising administering to the subject an agent comprising: at least 30 consecutive amino acids of a p62/SQSTM1 polypeptide or a variant thereof; a p62/SQSTM1 encoding nucleic acid, wherein said p62/SQSTM1 encoding nucleic acid encodes at least 30 consecutive amino acids of a p62/SQSTM1 polypeptide or a variant thereof; a p62/SQSTM1 polypeptide at least 90% identical to SEQ ID NO. 2; a p62/SQSTM1 polypeptide with at least one domain deletion; a p62/SQSTM1 nucleic acid encoding a polypeptide at least 90% identical to SEQ ID NO. 2; or a p62/SQSTM1 nucleic acid encoding a polypeptide with at least one domain deletion.

In some embodiments, the administering of the agent to the subject is via a carrier.

In some embodiments, the carrier comprises a microorganism, virus, nanoparticle, polymer, liposome, or a protein.

In some embodiments, the administering of the agent to the subject is via an intraocular, intramuscular, subcutaneous, per os, per rectum, intranasal, or intradermal route of administering.

Some embodiments further comprise selecting the subject based on signs or symptoms of age-related macular degeneration.

Some embodiments further comprise selecting the subject based on age or clinical signs or symptoms of aging.

Some embodiments further comprise selecting the subject based on presence of risk factors of age-related macular degeneration.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIG. 1 shows a wild type nucleic acid sequence of human p62 (SEQ ID NO: 1);

FIG. 2 shows a wild type amino acid sequence of the human p62/SQSTM1 encoded by the nucleic acid sequence (SEQ ID NO: 2);

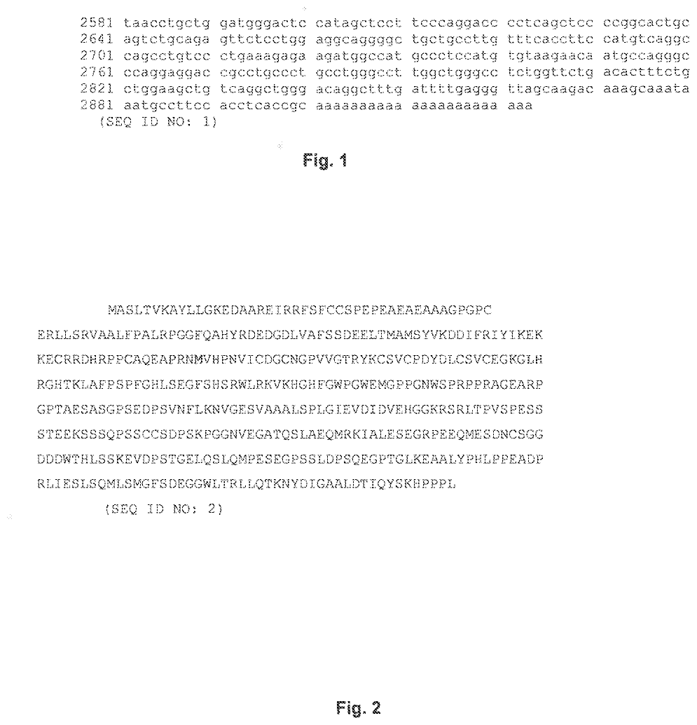

FIGS. 3A-B show the effect of treatment with p62DNA on the retinopathy developing in OXYS rats at 1.5 and 12 months of age;

FIGS. 4A-B show the effect of p62DNA on development of retinopathy and the effect persisted for 6 months after the last injection;

FIGS. 5A-D show the effect of p62DNA on expression of retinal p62;

FIGS. 6A-B show the effect of p62DNA on degeneration of neuroretina and RPE.

All illustrations of the drawings are for the purpose of describing selected versions of the present invention and are not intended to limit the scope of the present invention.

### DETAILED DESCRIPTION

Senescence-accelerated OXYS rats spontaneously develop a phenotype similar to human AMO-like retinopathy. Retinopathy that develops in OXYS rats even at a young age corresponds (in terms of clinical manifestations and morphological characteristics) to the dry atrophic form of AMO in humans. Furthermore, neovascularization develops in some (˜10-20%) of these rats with age. The clinical signs of AMO-like retinopathy appear by the age of 3 months in 100% of OXYS rats against the background of a reduction in the transverse area of the RPE and impairment of choroidal microcirculation. Significant pathological changes in the RPE as well as clinical signs of advanced stages of retinopathy are evident in OXYS rats older than 12 months. These changes manifest themselves as excessive accumulation of lipofuscin and amyloid in the retinal pigment epithelium (RPE) regions, disturbances in the morphology of the RPE sheet, including an increase in the proportion of multinucleated cells, hypertrophy, distortion of cell shape, and reactive gliosis. This rat model is successfully used to study the pathways and molecular alterations implicated in the development and progression of age-related diseases as well as to test new therapeutic interventions.

### p62DNA Inhibits Retinopathy Development in OXYS Rats

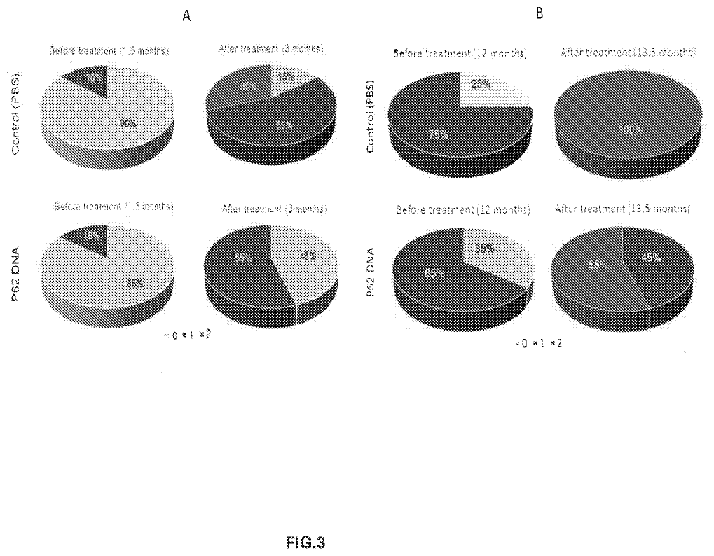

We assessed possible prophylactic effects of p62DNA against the development of retinopathy. A set of six p62DNA weekly injections started at the age of 1.5 months prior to any signs of retinopathy. Preliminary examination of rats at the age of 1.5 months showed that in experimental and control groups of OXYS rats signs of the first stage (1 arbitrary unit (a.u.)) of retinopathy were present in 15 and 10% of animals, respectively. Five injections of the p62DNA ones a week (from 1.5 months of age) significantly slowed down development of retinopathy in OXYS rats (FIG. 3). By the age of 3 months, 55% of the eyes in the control group developed the signs of the first stage of retinopathy, 30% developed the second stage, and only 15% of the eyes remained without the signs of the disease. In contrast, in the p62DNA treated group, 55% of the eyes developed the signs of the first stage of retinopathy, while the other 45% of the eyes did not show any signs of degeneration. Accordingly, a statistical analysis showed that the average level of retinopathy in the p62DNA-treated OXYS rat's eyes was 2.5 times lower than in the control animals (0.45±0.11 and 1.15±0.15 a.u., p<0.001, respectively).

Another study was tested the plasmids' effects on progression of AMO in the older animals. Examination of older animals at the age of 12 months revealed that all animals had signs of retinopathy in at least one eye. 75% of the eyes in the control group manifested changes corresponding to the AMO predisciform stage (1 a.u.) and 25% did not have the signs of retinopathy. In the experimental group, 65% of the eyes had changes corresponding to the predisciform stage (1 a.u.) and 35% of rats did not have the signs of retinopathy. Statistical analysis showed that retinopathy continued to progress in both control and experimental groups but p62DNA reduced the severity of pathological changes in the eyeground of OXYS rats (p<0.001). By the time of the second eye inspection at the age of 13.5 months, all the eyes in the control group had signs of retinopathy corresponding to the second stage of AMO (2 a.u.). At the same time, the p62DNA-treated OXYS rats demonstrated pathological changes corresponding to the first stage of AMO in the 45% of the retinas, and to the second stage in the 55% of the eyes. These data indicate that administration of p62DNA in the prophylactic setting significantly delays development of AMO signs and alleviates the severity of the disease.

### The Effect of p62DNA Persisted for 6 Months after the Treatment

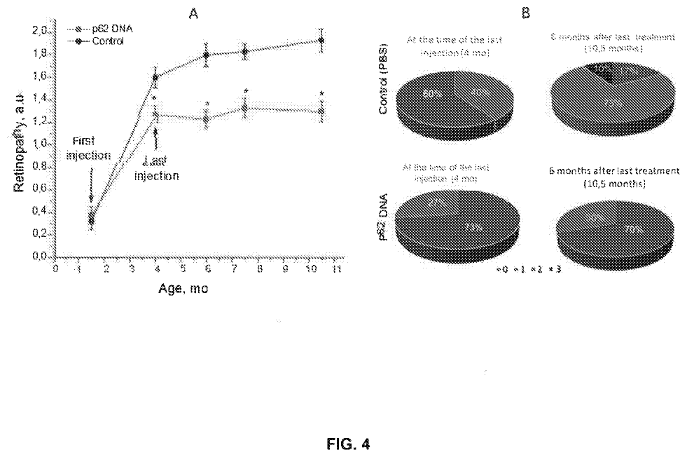

To assess the duration of the effect of p620NA on AMO, groups of OXYS rats were administered weekly injections of either p62DNA or PBS control and then observed for 6 months. The first injection took place at the age of 1.5 month, and the last one at 4.5 months. Each animal was examined by an ophthalmologist every second week. The results of examination are shown in FIG. 4. The first (preliminary) examination of rats at the age of 1.5 months revealed that the same percentage of eyes in the experimental and the control group of OXYS rats had signs of the first stage of retinopathy (30% and 35% respectively). At the age of 4 months, 73% of the eyes manifested signs of first stage retinopathy, and 27% manifested signs of the second stage disease in p62-treated rats. At the same time in control rats, we found signs corresponding to the first and second stages of the disease in 40% and 60% of the eyes respectively. According to the ANOVA analysis, an averaged stage of retinopathy in p62-treated rats was significantly reduced compared to the control (p<009).

Starting with 4 months of age, p62DNA completely prevented further development of retinopathy in OXYS rats. As a result, the severity of retinopathy signs at the age of 10.5 months remained at the level of the 4-month old animals: 70% of the eyes of OXYS rats from this group had signs of the first-stage and 30%, of the second stage of retinopathy indicating that the disease remained stable during at least 6 months following the p62DNA injections. In contrast, examination of the control animals at the ages of 7.5 and 10.5 months indicated enhancements of the severity of pathological changes (p<0.015). At the age of 10.5 months, we detected signs of the first-stage AMO in 17% of the eyes, the second-stage AMO in 73% of the eyes, and the third-stage AMO in 10% of the eyes of the control OXYS rats (FIG. 4). Therefore, administration of p62DNA precludes further disease progression, an effect that can last for 6 months.

### Administering of p62DNA does not Change Expression of Retinal p62

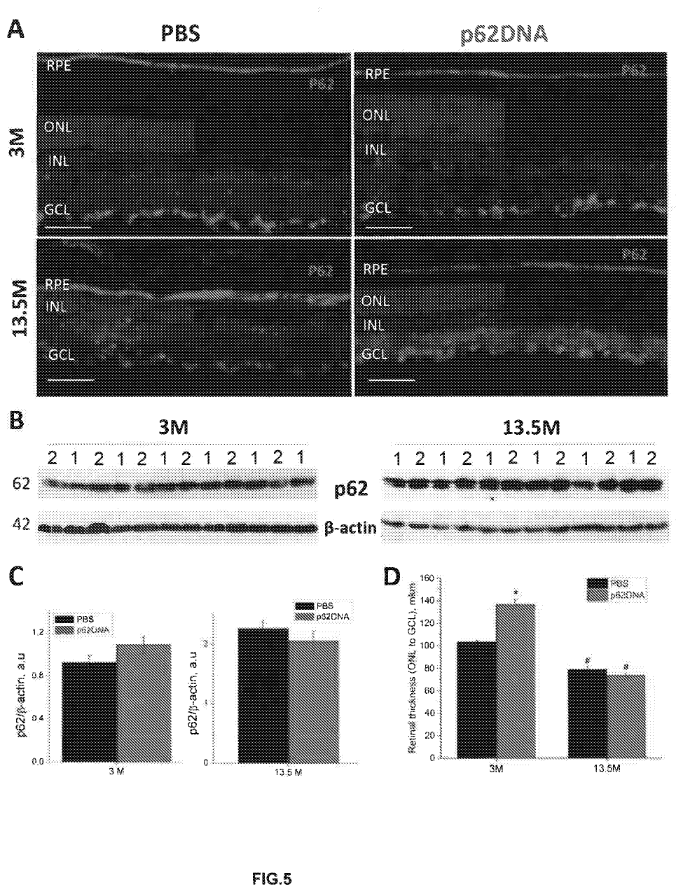

Western blot analysis and immunohistochemistry were performed to determine expression of p62 in the retina of 3- and 13.5-month-old OXYS rats receiving injections of PBS (vehicle control) or p62DNA (FIG. 5A-C). Immunohistochemical staining of the retinal slices revealed strong p62 expression in the RPE cells and around the nuclei of the inner nuclear (INL) and ganglion cells layers (GCL) in both control and treated animals. P62 expression was weaker in outer and inner plexiform layers (OPL and IPL) in rats of all groups (FIG. 3A) (n=4 p62DNA, n=4 PBS). Also, immunostaining revealed p62 granules in plexiform layers, and the number of these granules increased with age. However, we did not detect any significant differences in the p62 immunostaining between the plasmid-treated and PBS groups. The lack of difference in expression of p62 protein in rat retina in control and following p62DNA administration either at 3 months or 13.5 months old animals was further confirmed by immunoblotting with anti-p62 antibody (FIGS. 5B-C) (n=6 p62DNA, n=6 PBS).

### Administering p62DNA Prevents Degeneration of Neuroretina and RPE

We observed a higher overall retinal thickness (from GCL to ONL) in young OXYS rats treated with p62DNA compared to PBS-treated group (FIG. 5D) (p<0.05, n=4 p62DNA, n=4 PBS). In the control OXYS rat group, we observed a substantial reduction of the number of rows of photoreceptors (FIG. 5A) and the retinal thickness (FIG. 5D) by 13.5 months of age. These changes indicate progressive retinal neurodegeneration. At the same time, the age-associated reduction of the number of photoreceptor rows observed at the age of 13.5 months was substantially smaller in animals that received 6 weekly injections of p62DNA from 12 months.

However, treatment of the older rats with the plasmid (starting at the age of 12 months) did not prevent or reverse the decline in retinal thickness.

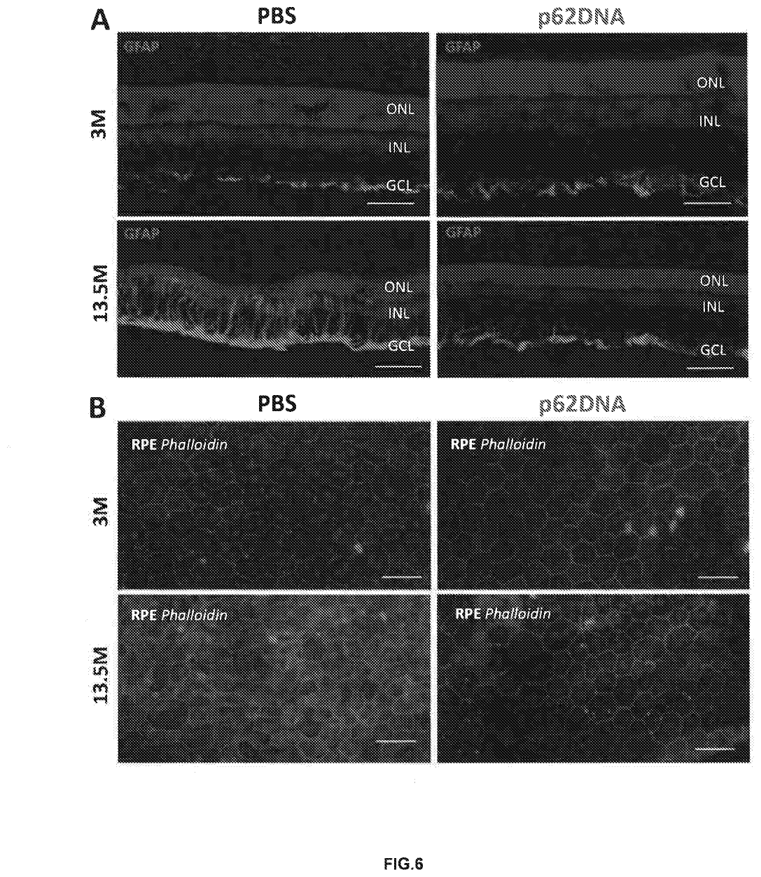

RPE cells are first affected during AMO pathogenesis. In line with this observation, destructive alteration in RPE cells is a primary change during the development of retinopathy in OXYS rats. We investigated the effect of p62DNA on the state of actin cytoskeleton in RPE cell by staining RPE flat mounts with phalloidin (FIG. 6B). According to the commonly accepted practice assuring the highest quality and reproducibility, we analyzed only the central zone of the RPE, which is in close proximity to the exit site of the optic nerve. In 3-months old PBS control OXYS rats, we observed hypertrophic and multinucleate RPE cells with loss of hexagon shape, indicating significant abnormalities. By the age of 13.5-months in PBS control OXYS rats, the majority of RPE cells displayed disorganized morphology and the loss of hexagon shape. This qualitative assessment showed a significant increase in proportion of multinucleate and hypertrophic RPE cells upon aging. In contrast, in p62DNA treated groups, the RPE cell exhibited mostly a regular organized structure with a smaller proportion of pathologically altered cells (FIG. 6B). Thus, the p62DNA treatment significantly alleviated destructive alterations of RPE cells.

### p62DNA Reduces Upregulation of GFAP Expression

Upregulation of glial fibrillary acidic protein (GFAP) is a well-established indicator of retinal injury and reactive gliosis. We investigated expression of GFAP by immunohistochemistry with the corresponding antibody. At the age of 3 months, GFAP staining was mainly confined to astrocytes and ganglion cell layer at the inner limiting membrane (FIG. 4A), and there was no significant difference between levels of GFAP in the retina of p62DNA and PBS groups. By the 13.5 months of age, astrocytes and Muller cells were strongly activated in control animals, as shown by the intense GFAP staining in the macroglial outgrowths from GCL towards the outer limiting membrane beyond ONL, representing massive gliosis (FIG. 6A). However, the p62DNA treatment strongly reduced, and in some cases completely prevented GFAP upregulation in 13.5-month-old OXYS rats (FIG. 6A) (n=4 p62DNA, n=4 PBS). Therefore, administration of p62DNA has a strong preventive effect on multiple hallmarks of developing AMO.

### EXAMPLE 1. p62DNA INHIBITS RETINOPATHY DEVELOPMENT IN OXYS RATS

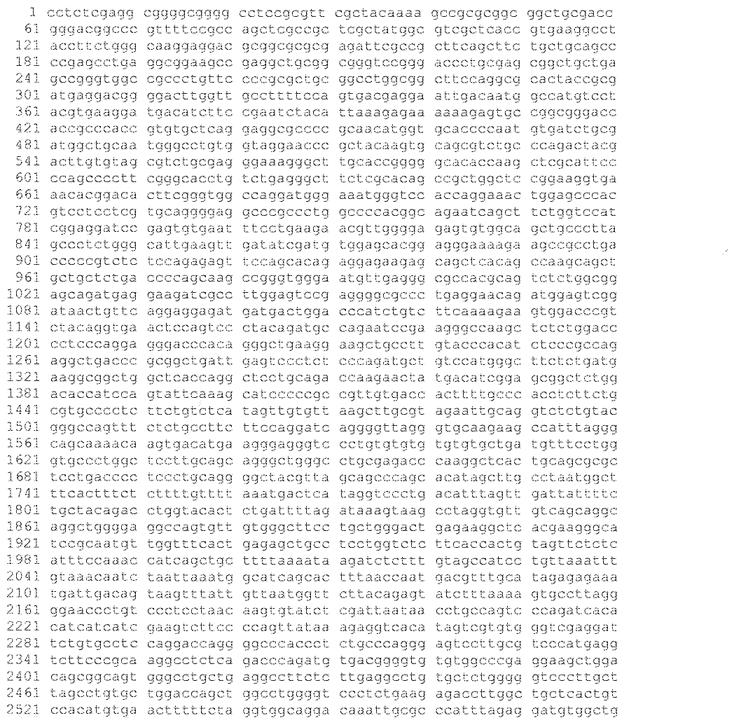

DNA plasmid used is a human p62 (SQSTM, isoform 1, FIG. 1) -encoding DNA vaccine (Elenagen) was produced using EndoFree Plasmid Giga Kit (Qiagen).

Male senescence-accelerated OXYS rats were obtained from the Center for Genetic Resources of Laboratory Animals at the Institute of Cytology and Genetics, the Siberian Branch of the Russian Academy of Sciences The OXYS strain was derived from the Wistar strain of rats at the Institute of Cytology and Genetics. At the age of 4 weeks, the pups were weaned, housed in groups of five animals per cage (57×36×20 cm) and kept under standard laboratory conditions (22° C.±2° C., 60% relative humidity, and 12-hour light/12-hour dark cycle; lights on at 9 a.m.). The animals were provided with standard rodent feed (PK-120-1; Laboratorsnab, Ltd., Moscow, Russia) and water ad libitum.

OXYS rats at the age of 1.5 months (n=20) and 12 months (n=20) were divided into four groups (n=10) and were injected intramuscularly (femoral quadriceps) with p62DNA, 150 μg per rat in 60 μl (Elenagen, 2.5 mg/ml) of phosphate-buffered saline (PBS) or with only PBS. All groups were subjected to five injections at one-week intervals. Ophthalmoscopic examination was carried twice: before and 2 weeks after the last plasmid injection. The rats were euthanized using CO₂ inhalation and decapitated 8 days after the last examination of eyes. Eyes from four rats per group were used for immunohistochemistry (the right eyes) and RPE flat-mount staining (the left eyes). At least four tissue slices were analyzed per animal.

All rats underwent funduscopy with a Heine BETA 200 TL Direct Ophthalmoscope (Heine, Herrsching, Germany) after dilatation with 1% tropicamide. An assessment of stages of retinopathy was performed according to the Age-Related Eye Disease Study grade protocol (AREDS; http://eyephoto.ophth.wisc.edu). The degree of retinopathy was estimated as follows: 0 arbitrary units (a.u.) corresponds to healthy retina; 1 a.u.—appearance of drusen and other pathological changes in the RPE and partial atrophy of the choroid capillary layer; 2 a.u.—exudative detachment of RPE and of retinal neuroepithelium, with further choroid capillary layer atrophy; and 3 a.u.—neovascularization and exudative-hemorrhagic detachment of RPE and neuroepithelium scarring.

FIG. 3 shows the effect of treatment with p62 plasmid on the retinopathy developing in OXYS rats at 1.5 and 12 months of age. The data are presented as percentage of eyes with stages (0, 1, and 2) of retinopathy before and after treatment in control (PBS) and p62-treated OXYS rats. In each group, 20 eyes of 10 animals were examined.

### EXAMPLE 2. EFFECT OF p62DNA REMAINED FOR 6 MONTHS AFTER THE TREATMENT

1.5-month old OXYS rats were randomly divided into two groups (n=15) and were injected intramuscularly (femoral quadriceps) with p62DNA 150 μg per rat in 60 μl (Elenagen, 2.5 mg/ml) on PBS or with only PBS (n=15). All groups were subjected to nine injections at one-week intervals. The animals received the last injection at the age of 4 months. An ophthalmologist examined all animals five times: before treatment at the age 1.5 months and at the ages 4, 6, 8, and 10.5 months, respectively.

FIG. 4 demonstrates that p62DNA suppresses development of retinopathy and the effect persisted for 6 months after the last injection. (A). The data are presented as a.u. corresponding to the stages of retinopathy. A significant increase in the severity of retinopathy according to the pairwise comparisons of the eye condition before and after treatment. (B) Stages of retinopathy in 4- and 10.5-month-old controls and p62 treated OXYS rats. Treatment was started at the age 1.5 months. In each group, 30 eyes of 15 animals were examined. The data are presented as the percentage of eyes with stages (0, 1, 2 and 3) of retinopathy. The data were analyzed using repeated-measures ANOVA (analysis of variance) and nonparametric tests using a statistical package (Statistica 8.0 software). A one-way analysis of variance was used for individual group comparisons. The Newman-Keuls test was applied to significant main effects and interactions in order to assess the differences between some sets of means. To assess the therapeutic effectiveness, we performed a dependent pairwise comparison of the eye states before and after treatment (t-test for dependent samples). The data are presented as mean±SEM. The differences were considered statistically significant at p<0.05.

### Example 3. p62DNA DOES NOT CHANGE EXPRESSION OF RETINAL p62

To measure p62 protein levels retinas obtained from six rats for each group was used (the left and right eyes were mixed). The retina was separated from other tissues, placed in microcentrifuge tubes for protein isolation, and frozen in liquid nitrogen. All specimens were stored at −70° C. before the analysis.

The frozen tissues of retina were homogenized in protein lysis buffer radioimmunoprecipitation assay (50 mmol/L Tris-HCI, pH 7.4; 150 mmol/L NaCl; 1% Triton X-100; 1% sodium deoxycholate; 0.1% SOS; and 1 mmol/L EDTA) supplemented with protease inhibitor cocktail (P8340; Sigma-Aldrich, St. Louis, Mo.). After incubation for 20 minutes on ice, samples were centrifuged at 9660×g at 4° C. for 30 minutes, and the supernatants were transferred to new tubes. Total proteins were measured with a Bio-Rad Bradford kit (Bio-Rad Laboratories). Immunoblotting was performed. Samples were resolved on 12% SOS-PAGE on Tris-glycine buffer (25 mmol/L Tris base, 190 mmol/L glycine, and 0.1% SOS) and transferred to nitrocellulose membranes. Antibodies and dilutions used in immunoblotting included an anti-P62 antibody (1:1,000) and anti-β-actin antibody (1:1,000). After blockage with 5% bovine serum albumin (BSA; Sigma-Aldrich) in 0.01 M phosphate buffer with 0.1% Tween-20 (PBS-T) for 1 hour, the membranes were incubated with the primary antibodies at 4° C. overnight. After incubation with the respective secondary antibody (1:3,000), chemiluminescent signals were measured and scanned, and intensity of the bands was quantified in the ImageJ software (NIH, Bethesda, Md., USA). β-actin served as an internal loading control.

Immunofluorescent staining was performed according to a standard method. The eyes were removed and fixed in fresh 4% paraformaldehyde in PBS for 2 hours, washed three times in PBS, and then cryoprotected in graded sucrose solutions (10%, 20%, and 30%).

Posterior eyecups were embedded in Tissue-Tek® O.C.T. Compound (Sakura), frozen, and stored at −80° C. Tissue slices (10 μm thick) were prepared on a Microm HM-505N cryostat at −20° C., transferred onto Polysine® glass slides (Menzel-Glaser), and stored at −20° C. Primary antibodies and dilutions were as follows: anti-Iba1 (1:250), anti-Gfap (1:250), and anti-Cd68 (1:300). Primary antibodies were incubated for 18 hours at 4° C. After incubation with the respective secondary antibodies diluted 1:300 for 1 hour at room temperature, the tissue slices were coverslipped with the mounting medium containing DAPI (ab104139, Abeam) and were examined under the Zeiss microscope Axioplan 2. The negative control samples with the omitted primary antibody emitted only a minimal autofluorescent signal. For each image acquisition, all imaging parameters were the same. The morphometric parameters were measured using quantitative analyses of the images performed with Axiovision software (SE64 4.9.1). Estimation was performed by examination of the five fields of view for each retina. Mouse monoclonal anti-p62 (ab56416), rabbit polyclonal anti-actin (ab1801), and a secondary antibody—a donkey anti-goat IgG H&L antibody (conjugated with Alexa Fluor® 488; ab150129), donkey anti-rabbit IgG H&L antibody (conjugated with Alexa Fluor® 488; ab150073), donkey anti-mouse IgG H&L antibody (conjugated with Alexa Fluor® 568; ab175472) and goat anti-rabbit IgG H&L antibody (HRP; ab6721) were acquired from Abeam (Cambridge, UK).

FIGS. 5A-D shows the effect of p62 DNA on endogenous p62 expression and retinal thickness in the retina of OXYS rats at 3 and 13.5 months. FIG. 5A shows representative p62 immunofluorescence of retinal cryosections from 3- and 13.5-month-old OXYS rats treated by PBS (left) or p62DNA plasmid (right). Scale bar: 50 RPE: retinal pigment epithelium; ONL: outer nuclear layer; INL: inner nuclear layer; GCL: ganglion cells layer. FIG. 5B shows representative immunoblots of p62 in the retina of OXYS rats. 1—PBS; 2—p62DNA. FIG. 5c shows levels of p62 protein by immunoblot. FIG. 5D shows measurements of retinal thickness (from ONL to GCL) in 3-and 13.5-month-old OXYS rats treated by p62 DNA or PBS. p<0.05, statistically significant effect of p62DNA; #p<0.05 between 3 and 13.5 months. Data are presented as mean±SEM.

### EXAMPLE 4. p62DNA PREVENTS DEGENERATION OF NEURORETINA AND RPE

RPE flat-mount staining was done as described below. Enucleated eyes with an incision along the limbus were fixed in 4% paraformaldehyde in phosphate-buffered saline (PBS) for 2 hours as described by previous work. The anterior segment of the eye (cornea, iris, ciliary body, and lens) was removed. Retinal tissue was carefully excised from the eyecup, and the remaining cups containing RPE, choroid, and sclera were thoroughly washed in PBS with 0.1% Triton X-100 (PBST) and dissected into quarters by radial cuts. The RPE/choroid flat mounts were incubated in PBS/bovine serum albumin (BSA) 5% with 1% Triton X-100 for 1 hour for blocking and permeabilization. Next, the samples were stained with fluorescein isothiocyanate (FITC)-phalloidin (1:100, P5282, Sigma-Aldrich) at 4° C. for 48 hours to visualize the cytoskeleton and cell shapes during en face imaging. After washes in PBST, the RPE/choroids were flat-mounted on glass slides and were coverslipped with the Fluoro-shield mounting medium containing 4′,6-diamidino-2-phenylindole (DAPI; ab104139, Abeam). Images were acquired with a confocal microscope (LSM 780 NLO, Zeiss).

FIGS. 6A-B show the effect of p62DNA on the GFAP expression and the state of RPE cells. FIG. 6A shows representative GFAP immunostaining in retina of 3- and 13.5-month-old OXYS rats treated with PBS (left) or p62DNA (right). GFAP staining was mainly confined to astrocytes and the ganglion cell layer at the inner limiting membrane in OXYS rats at the age of 3 months. In the PBS-treated 13.5-month-old OXYS rats, the increased GFAP expression was observed along the Muller glial cell processes extending towards the outer limiting membrane, representing massive gliosis. p62DNA treatment prevented GFAP accumulation in 13.5-month-old OXYS rats. Scale bar: 50 μm. ONL: outer nuclear layer; INL: inner nuclear layer; GCL: ganglion cells layer. FIG. 6B shows representative images of phalloidin-stained RPE flat-mounts of 3- and 13.5-month-old OXYS rats, treated with PBS (left) or p62DNA (right). p62DNA treatment slowed down development of the destructive alterations of RPE cells (the loss of regular hexagonal shape, the hypertrophy, and the multinucleation) in OXYS rats. Scale bar: 50 μm.

Although the invention has been explained in relation to its preferred embodiment, it is to be understood that many other possible modifications and variations can be made without departing from the spirit and scope of the invention as hereinafter claimed. While this invention has been particularly shown and described with references to preferred embodiments thereof, it will be understood by those skilled in the art that various changes in form and details may be made therein without departing from the scope of the invention encompassed by the appended claims.

1. A method of preventing, treating, alleviating, ameliorating, relieving, delaying onset of, inhibiting progression of, reducing severity of, or reducing incidence of one or more symptoms of age-related macular degeneration in a subject comprising administering to the subject an agent comprising:
at least 30 consecutive amino acids of a p62/SQSTM1 polypeptide or a variant thereof;
a p62/SQSTM1 encoding nucleic acid, wherein said p62/SQSTM1 encoding nucleic acid encodes at least 30 consecutive amino acids of a p62/SQSTM1 polypeptide or a variant thereof;
a p62/SQSTM1 polypeptide at least 90% identical to SEQ ID NO. 2;
a p62/SQSTM1 polypeptide with at least one domain deletion;
a p62/SQSTM1 nucleic acid encoding a polypeptide at least 90% identical to SEQ ID NO. 2; or
a p62/SQSTM1 nucleic acid encoding a polypeptide with at least one domain deletion.

2. The method of claim 1, wherein the administering the agent to the subject is performed in combination with a second agent preventing or treating age-related macular degeneration.

3. A method of preventing, reversing, reducing, or modulating intraocular vascularization, oxidative stress, autophagy, or inflammation in a subject comprising administering to the subject an agent comprising:
at least 30 consecutive amino acids of a p62/SQSTM1 polypeptide or a variant thereof;
a p62/SQSTM1 encoding nucleic acid, wherein said p62/SQSTM1 encoding nucleic acid encodes at least 30 consecutive amino acids of a p62/SQSTM1 polypeptide or a variant thereof;
a p62/SQSTM1 polypeptide at least 90% identical to SEQ ID NO. 2;
a p62/SQSTM1 polypeptide with at least one domain deletion;
a p62/SQSTM1 nucleic acid encoding a polypeptide at least 90% identical to SEQ ID NO. 2; or
a p62/SQSTM1 nucleic acid encoding a polypeptide with at least one domain deletion.

4. The method of any of the claims 1-3 wherein the administering of the agent to the subject is via a carrier.

5. The method of claim 4 where the carrier comprises a microorganism, virus, nanoparticle, polymer, liposome, or a protein.

6. The method of any of the claims 1-3, wherein the administering of the agent to the subject is via an intraocular, intramuscular, subcutaneous, per os, per rectum, intranasal, or intradermal route of administering.

7. The method of any of the claims 1-3 further comprising selecting the subject based on signs or symptoms of age-related macular degeneration.

8. The method of any of the claims 1-3 further comprising selecting the subject based on age or clinical signs or symptoms of aging.

9. The method of any of the claims 1-3 further comprising selecting the subject based on presence of risk factors of age-related macular degeneration.

10. The method of any of the claims 1-3 wherein a nucleic acid is a plasmid or an RNA.
